(19) United States
(12) Patent Application Publication
(10) Pub. No.: US 2026/0069189 A1
(43) Pub. Date: Mar. 12, 2026

(54) CARDIAC SIGNAL PROCESSING DEVICE

(71) Applicant: SUBSTRATE HD, Marseille (FR)

(72) Inventors: Thomas BOUDOU, Marseille (FR); Céline HAJJAR, Marseille (FR)

(73) Assignee: SUBSTRATE HD, Marseille (FR)

(21) Appl. No.: 19/107,843

(22) PCT Filed: Aug. 24, 2023
(86) PCT No.: PCT/FR2023/051289

§ 371 Date: Feb. 28, 2025

(30) Foreign Application Priority Data
Sep. 2, 2022 (FR) FR2208851

(51) Int. Cl. A61B 5/353

(52) U.S. Cl. CPC A61B 5/353

(57) ABSTRACT

A device for processing cardiac signals including a memory receiving input data sets including a plurality of P wave segments associated with an electrocardiogram track and with an acquisition time window, and a plurality of coronary sinus signals associated with the same acquisition time window and having one or more activation sequence(s). The device includes an extractor arranged, for a given input data set, to determine for at least some P wave segments of the given input data set a wave polarity profile type, at least one extremum feature. The extractor is also arranged to determine, for each group of track P wave data, a set of track P wave features comprising a polarity profile type, a data set extremum feature value for each calculated extremum feature type and an integral value determined based on the data of the corresponding group of track P wave data.

### CROSS-REFERENCE TO RELATED APPLICATION(S)

This application is a 35 U.S.C. § 371 National Stage of International Patent Application No. PCT/FR2023/051289, filed on Aug. 24, 2023 claiming priority to and the benefit of French Patent Application No. 2208851, filed on Sep. 2, 2022, the disclosure of which is incorporated herein by reference in its entirety.

### TECHNICAL FIELD

The invention relates to the field of cardiac signal processing. More specifically, it finds application in the field of atrial fibrillation treatment.

### BACKGROUND

In general, the treatment of atrial tachycardia (or “AT”) consists in burning the area of the heart at the origin of the tachycardia. For this purpose, this area should be detected at first.

Most research articles giving an overview on the problem of location of the AT are primarily based on the analysis of the “P waves”, a specific portion of the electrocardiogram (hereinafter “ECG”) which corresponds to the depolarisation of the auricles.

In 1995, in the article “Use of p wave configuration during atrial tachycardia to predict site of origin”, Journal of the American College of Cardiology, 26(5):1315-1324, Tang et al have focused on the analysis of the polarity of the P waves in the surface electrodes to determine which auricle (the right one or the left one) is at the origin of the tachycardia. The derivations aVL and V1 have proven to be the most useful ones to differentiate the right sites from the left sites: a positive P wave in the aVL derivations predicts a right site with a 88% sensitivity and a 79% specificity. The sensitivity and the specificity of a positive P wave in the V1 derivation predicting a left site have been 93% and 88% respectively.

Since then, several studies have extended the analysis of the polarity of the P waves to predict the location of the site with a greater accuracy. In 2006, in the article “P-Wave Morphology in Focal Atrial Tachycardia: Development of an Algorithm to Predict the Anatomic Site of Origin», 48:1010-1017, Kistler et al have studied 130 focal AT to build a decision tree in order to find the origin amongst 11 possible regions, where the nodes are divided according to the polarity of the P waves in the different ECG derivations. This algorithm has succeeded in correctly classifying the origin in 93% of the 30 new TAs.

However, the criteria retained in these articles prevent use thereof in a real-time context. Indeed, they are based on a posteriori processing allowing detecting the P waves in a very accurate manner, which is not possible in real-time. Based on the P waves determined in real-time, the results would be less conclusive and these methods unsuitable.

### SUMMARY OF THE INVENTION

The invention improves the situation. To this end, it provides a device for processing cardiac signals, comprising a memory arranged to receive input data sets each comprising a plurality of P wave segments each associated with an electrocardiogram track and with an acquisition time window, and a plurality of coronary sinus signals associated with the same acquisition time window and having one or more activation sequence(s), an extractor arranged, for a given input data set, to determine for at least some P wave segments of the given input data set a wave polarity profile type, at least one extremum feature of a type selected from a group of types comprising the number of positive local extrema, the number of negative local extrema, the positive prominence maximum and the negative prominence maximum, and at least one integral value of these P wave segments, and to associate the resultant data into a group of track P wave data as a function of the electrocardiogram track with which each P wave segment is associated based on which said resultant data have been calculated. The extractor is further arranged to determine, for each group of track P wave data, a set of track P wave features comprising a polarity profile type, a data set extremum feature value for each calculated extremum feature type and an integral value determined based on the data of the corresponding group of track P wave data. The extractor is further arranged to determine activation times in at least some of the activation sequences of the coronary sinus signals of the given input data set and to deduce a set of time values therefrom, and to return a set of data set features comprising on the one hand the set of time values and, on the other hand, the sets of track P wave features. The device also comprises a machine-learning based locator using decision trees arranged to receive a set of data set features as input, and to return a cardiac region identifier as output.

This device is particularly advantageous because it allows determining in real-time a probable area of the anomaly at the origin of the perpetuation of the TA. This means that a practitioner has the possibility of focussing on a reduced portion of the heart to determine which he/she considers the actual area at the origin of the TA. Thus, the determination of the probable origin area of the AT enables the practitioner to save a valuable time, although the latter has neither a confirmed medical sense nor the value of a diagnosis.

According to various embodiments, the invention may have one or more of the following features:
the locator is arranged to implement a random-forest classifier,
the extractor is arranged to determine a data set extremum feature value indicating an absence of determination for a non-calculated extremum feature type, and to return a set of data set features comprising a data set extremum feature value for each extremum feature type,
the extractor is arranged to determine a data set extremum feature value for each type of the group of types,
the extractor is arranged, for a given set of track P wave features, to determine the polarity profile type while retaining the predominant wave polarity profile type in the corresponding group of track P wave data,
the extractor is arranged, for a given set of track P wave features, to determine the data set extremum feature value for each calculated extremum feature type and the integral value based on the average of these values in the corresponding group of track P wave data,
the extractor is arranged to calculate the set of time values based on the difference between the activation times of the activation sequences of the coronary sinus signals, and
the locator is arranged to receive as input a set of data set features comprising 9 sets of track P wave features, and a set of time values comprising 4 values.

The invention also relates to a method for processing cardiac signals comprising the following operations:
a) receiving input data sets each comprising a plurality of P wave segments each associated with an electrocardiogram track and with an acquisition time window, and a plurality of coronary sinus signals associated with the same acquisition time window and having one or more activation sequence(s),
b) for a given input data set, for at least some of the P wave segments of the given input data set:
b1) determining a wave polarity profile type,
b2) determining at least one extremum feature of a type selected from a group of types comprising the number of positive local extrema, the number of negative local extrema, the positive prominence maximum and the negative prominence maximum, and
b3) determining at least one integral value of these wave segments P,
b4) associating the data resulting from the operations b1) to b3) into a group of track P wave data as a function of the electrocardiogram track with which each P wave segment is associated based on which said resultant data have been calculated,
b5) determining, for each group of track P wave data, a set of track P wave features comprising a polarity profile type, a data set extremum feature value for each calculated extremum feature type and an integral value determined based on the data of the corresponding group of track P wave data,
b6) determining activation times in at least some of the activation sequences of the coronary sinus signals of the given input data set and to deduce a set of time values therefrom,
b7) returning a set of data set features comprising on the one hand the set of time values of the operation b6), and, on the other hand, the sets of track P wave features of the operation b5), and
c) providing a set of data set features obtained in step b7) as input of a machine-learning based locator using decision trees, returning a cardiac region identifier as output.

According to various embodiments, the method may have one or more of the following features:
the operation b2) comprises determining a data set extremum feature value for each type of the group of types,
the operation b5) comprises, for a given set of track P wave features, determining the polarity profile type while retaining the predominant wave polarity profile type in the corresponding group of track P wave data,
the operation b5) comprises, for a given set of track P wave features, determining the data set extremum feature value for each calculated extremum feature type and the integral value based on the average of these values in the corresponding group of track P wave data, and
the operation b6) comprises calculating the set of time values based on the difference between the activation times of the activation sequences of the coronary sinus signals.

The invention also relates to a computer program comprising instructions to execute the method according to the invention, a data storage medium on which such a computer program is recorded and a computer system comprising a processor coupled to a memory, the memory having recorded such a computer program.

### BRIEF DESCRIPTION OF THE DRAWINGS

Other features and advantages of the invention will appear better upon reading the following description, with reference to examples given for illustrative and non-limiting purposes, with reference to the drawings wherein:

FIG. 1 shows a schematic diagram of a device according to the invention,

FIG. 2 shows an example of a function implemented by the device of FIG. 1,

FIG. 3 shows an example of P wave polarity profiles searched in the function of FIG. 2,

FIG. 4 shows an example of a P wave segment and the types of measured features,

FIG. 5 shows an example of coronary sinus segments and the time values that are deduced therefrom, and

FIG. 6 shows a division of the auricles into 21 areas.

### DETAILED DESCRIPTION

The drawings and the description hereinafter essentially contain elements of certain nature. Hence, they could not only serve to better understand the present invention, but also contribute to the definition thereof, where appropriate.

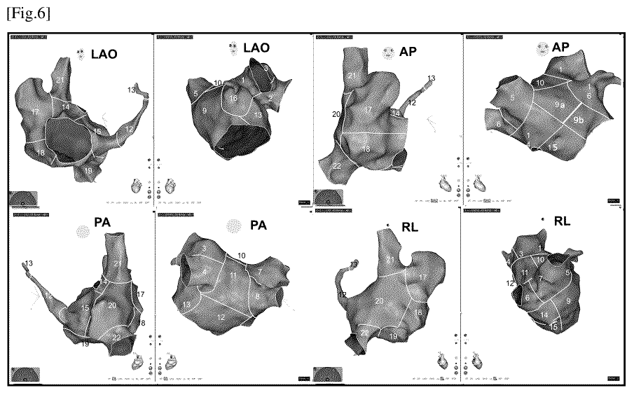

In attempt to classify the origin of the TA, the Applicant has for a long time worked based on a division of the auricles into 21 areas as shown in FIG. 6. Throughout these works, and searching for a solution that could be used in real-time, it has found that it were relevant to group these areas into 4 groups: the left auricle (areas 7, 8, 9A, 9B, 10, 11A, 11B and 12), the right auricle (areas 18, 19, 20B, 20H, and 22), the septum (areas 5, 6, 14, 15, 17 and 21), and the lateral portion of the left auricle (areas 1, 2, 3, 4, 13 and 16).

As this will be seen hereinbelow, the device of the invention allows determining, for a set of ECG signals and an intracardiac reference catheter commonly placed in the vein of the coronary sinus, which will hereafter be referred to by the expression “CS”, the group that contains the origin of the TA. This determination is particularly interesting because it offers a starting point to the practitioner to search and determine the specific origin area of the AT and therefore the diseased portion of the heart. Indeed, if these groups define 4 larger areas than the 21 original areas, this is already enough to confer a great advantage on the procedure.

Furthermore, the combination of ECG signals and of signals derived from the coronary sinus is particularly innovative. It has never been disclosed before, and it enables the use of machine learning, which enables the operation of the device 2 in real-time.

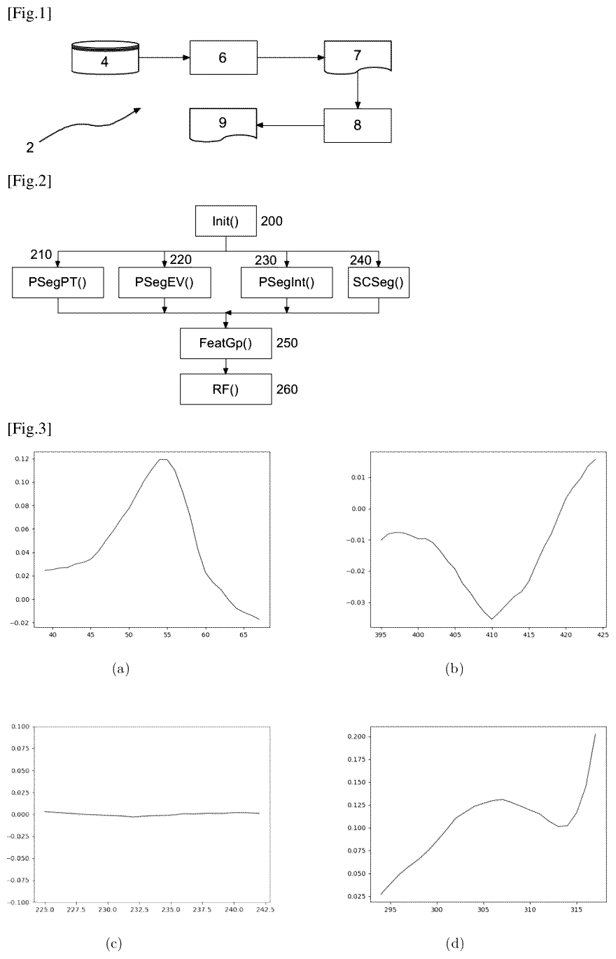

FIG. 1 shows a schematic diagram of a cardiac signal processing device 2 according to the invention. As shown in this figure, the device 2 comprises a memory 4, an extractor 6 and a locator 8. The extractor 6 determines a set of data set features 7 which will be described hereinbelow and which serves as input to the locator 8 which returns an origin area 9.

The memory 4 receives input data sets as input. Each input data set comprises a plurality of P wave segments each associated with an electrocardiogram track and with an acquisition time window, and a plurality of segments deduced from coronary sinus signals associated with the same acquisition time window. In concrete terms, this means that a time window, typically 10 seconds before a current measurement time, is used to split an ECG with 9 tracks (deduced from a 12-track sensor 3 of which are ignored as they correspond to a linear combination of some of the other tracks) as wells as 5 CS signal tracks. In the particular case of the ECG, only the segments corresponding to a P wave are used. Since each heart rate is particular, this means that the input data set receives a variable number of P wave segments each being deduced from an ECG track. The present invention does not cover the particular method for obtaining the P wave segments and assumes that these form an input. In some embodiments, the device 2 could be arranged to directly analyse the ECG tracks and to determine the P wave segments therein. By P wave segment, it should be understood that subsets of each ECG track are split inside the time window and define a segment of the latter.

As shown hereinbelow, all of the P wave segments of each data set are processed in order to deduce features therefrom which serve as an input to the locator 8. Thus, for each ECG track, 6 features have been determined, whereas 4 features are deduced from the CS signals. Hence, this results in a total of 58 features (6 features for 9 tracks plus 4) which form the input vector of the locator 8. Alternatively, the device 2 could determine that some P wave segments or CS signals are difficult to exploit and deduce no features for each of these but discard some of them. Still alternatively, the machine learning could be done on a smaller number of features, for example by determining only between 3 and 5 features per ECG track. Finally, the device 2 could also choose to keep only some ECG tracks (and therefore the corresponding P wave segments), and thus reduce the number of features.

The memory 4 may consist of any data storage type capable of receiving digital data: hard drive, solid-state drive, flash memory in any form, random-access memory, magnetic disk, distributed storage locally or in the cloud, etc.

In the example described herein, the memory 4 receives all data regarding the device 2, i.e. the programs and software instantiating the extractor 6 and the locator 8, the parameters and hyperparameters of these, the weights of the trees, the data sets received as input (where appropriate), the features determined by the extractor 6, the data stored in the buffer memory, as well as the origin area data at the output. The data calculated by the device may be stored on any type of memory similar to the memory 4, or on the latter. These data may be erased after the device has performed its tasks or kept.

The extractor 6 and the locator 8 directly or indirectly access the memory 4. They could be made in the form of an appropriate computer code executed on one or more processor(s). By processors, it should be understood any processor suited to the calculations described hereinbelow. Such a processor may be made in any known manner, in the form of a microprocessor for a personal computer, laptop, tablet or smartphone, an FPGA or SoC type dedicated chip, a computing resource on a grid or in the cloud, a cluster of graphical processors (GPUs), a microcontroller, or any other form capable of providing the computing power necessary to the completion of the process described hereinbelow. One or more of these elements may also be made in the form of specialised electronic circuits such as an ASIC. A combination of a processor and of electronic circuits may also be considered. Machine-learning dedicated processors could be used.

FIG. 2 shows an example of implementation of a function executed by the extractor 6 to extract the set of data set features 7 and to deduce the origin area 9 therefrom.

In an operation 200, the extractor 6 implements a function Init( ) which receives the current time point, and selects in the memory 4 the P wave segments which correspond to the time window defined by the current time point. Thus, as described hereinabove, the time window has as extreme ends the current time point on the one hand, and a time point located 10 seconds upstream of the current time point on the other hand. The Applicant has noticed that the 10 second duration allows obtaining a sufficient number of P wave segments to obtain a reliable origin area with current time points taken every 10 seconds. Alternatively, this duration could be different, for example a time window duration of 2 seconds, 5 seconds, or 15 seconds provided that at least one heartbeat has been detected for obtaining the P waves.

Thus, the function Init( ) extracts P wave segments and CS signals that correspond to a unique time window. Afterwards, four functions are executed in parallel to determine data that will allow producing the set of data set features 7.

In an operation 210, a function PSegPT( ) is executed to determine, for each P wave segment, a P wave polarity profile corresponding thereto. Indeed, the Applicant has determined that the P wave polarity profile is a significant indicator of the origin area of the TA. For this purpose, the function PSegPT( ) implements a dynamic time warping calculation (“Dynamic Time Warping” in English or “DTW”) in order to determine the similarity between each P wave segment and each amongst 4 P wave polarity profiles that the Applicant has identified as discriminating to determine the origin area of the TA. FIG. 3 shows an example of each of these 4 profiles.

Recall that the dynamic time warping is an algorithm allowing measuring similarity between two sequences of values that vary over time. Sequences of values are deformed by non-linear transformation of the time variable to determine a measurement of their similarity independently of some non-linear transformations of time. Thus, by comparing the DTW measurements between a given P wave segment and the 4 P wave polarity profiles, it is possible to retain that the P wave polarity profile for a given P wave segment is that one wherein the DTW between the two is the lowest. This amounts to consider that this P wave polarity profile is that to which the given P wave segment “most closely resembles”. Alternatively, if no P wave polarity profile seems to be the most relevant one, an “undetermined” type could be retained in order to improve the machine learning. Alternatively, the DTW could be replaced by the Euclidian distance, or another distance in mathematical terms to be minimised between the P wave and the 4 profiles. Another measurement of correlation with the profiles could also be used.

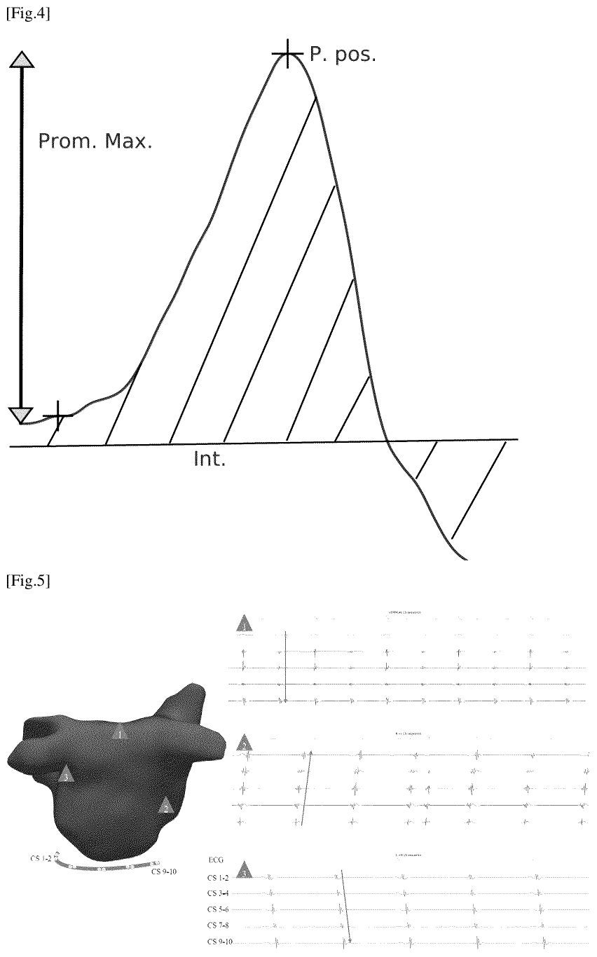

In an operation 220, a function PSegEV( ) is executed. This function is intended to extract for each P wave segment values that characterise its extrema (“peak” in English). Thus, as shown in FIG. 4, the relevant extremum values that could be retained are the number of positive extrema, the number of negative extrema, the maximum positive prominence, and the maximum negative prominence. These four features allow defining an image of each P wave segment, namely its number of positive and negative peaks (at which point the P wave segment is chaotic), as well as the magnitude of these peaks (in geography, the prominence is the altitude difference between a given summit and the highest saddle or pass allowing reaching an even higher summit, and this concept is of course extended in signal graphical analysis). Depending on the situations, as described hereinabove, only one type or two types of features could be calculated, i.e. for example only the number of positive peaks, or complementarily with the maximum positive prominence. In addition, some P wave segments could be discarded if their shape is not suited to these calculations.

In an operation 230, a function PSegInt( ) is executed. This function is intended to calculate for each P wave segment the integral of the corresponding signal. Indeed, this integral also characterises the measured force of the P wave. Herein again, some P wave segments could be discarded if this proves to be relevant. Alternatively, two integral values may be calculated: the integral of the positive portion and the integral of the negative portion, which together allow better describing the signal further. In this case, the set of data set features rather contains 67 inputs (58 values plus 9 more integral values).

Finally, in an operation 240, a function SCSeg( ) is executed. This function is intended to determine the activation time points within each coronary sinus track, and to return time values indicating the offsets between the activation start time points. Indeed, the sequence of the peaks in the CS signals may indicated whether the AT origin area is in the left or right auricle, and the Applicant has discovered that these features combine particularly well with the features deduced from the P wave segments in the context of a machine learning. FIG. 5 illustrates an example of CS signals acquired according to the location of the AT at places indicated by a triangle with an index 1, 2 or 3, on the left auricle, as well as the corresponding activation sequences (arrows on each acquisition). In the example described herein, all time values are considered with reference to the activation time point of the first coronary sinus signal. Alternatively, these values could be comprised between two successive signal activation time points (for example the difference between the first and second signals, the second and third signals, the third and fourth signals, and the fourth and fifth signals). What is important is that the measured time values are representative of the sequence of the peaks in the CS signals.

Although the operations 210, 220 and 230 have been described separately but executed by three distinct functions, they could be executed in sequence or in parallel within one single function. Similarly, the operation 240 could be carried out sequentially with respect to the operations 210 to 230, rather than in parallel. However, parallelising these operations has the advantage of being faster and therefore of promoting real-time execution.

However, a problem arises when executing a calculation by machine learning: as set out hereinabove, since it is the measurement time point (the current time point) which determines the time window, the number of P wave segments is not constant during the execution of the device 2. Beyond that, there is even a risk of over-representation of P wave segments associated with a particular ECG track compared to the others. Finally, if the number of P wave segments is not constant, it is not possible to define an input vector with a fixed dimension for machine learning.

Consequently, the function of FIG. 2 is carried on with an operation 250 in which a function FeatGp( ) is executed by the extractor 6. In a first step, this function groups together all of the data calculated by the operations 210 to 230 according to the ECG track from which the P wave segments at the origin of these values are deduced. This means that all of the P wave polarity profiles, the extremum values and the integral values associated with the same ECG track are grouped together into a group of track P wave data. Afterwards, in each group of track P wave data, a unique value is retained for each feature type, i.e. a P wave polarity profile, an extremum value for each type having been the object of a calculation, and an integral value. As mentioned before, preferably, it has four extremum values respectively corresponding to the number of (positive and negative) peaks and to the (positive and negative) maximum prominence. In the case of the P wave polarity profiles, a majority vote may be performed: for a given ECG track, it is the largest number of P wave polarity profiles of the P wave segments which designate the retained profile. In the case of the extremum and integral values, the average could be used. Other methods could be used: weighted average, median, regression, and minimum threshold for the majority vote.

Thus, 6 features are obtained for each ECG track, and they form sets of track P wave features. By grouping these sets with the set of time values deduced from the CS signals, the set of data set features comprising the aforementioned 58 features is obtained. In the case where the CS signals contain several activation sequences, the sets of values could be averaged in the operation 250 in a manner similar to the extremum values of the P wave segments.

This set could then be transmitted as an input vector to the locator 8 which executes a function RF( ) in an operation 260. The function RF( ) implements a random-forest classifier that has been trained with a set of training data in which the output origin area was known. Thus, thanks to training of the machine learning engine, the origin area 9 could be determined in real-time.

Alternatively, the locator 8 could implement a machine learning using decision trees different from random-forests, for example based on gradient boosting (“gradient boosting” in English), or extreme gradient boosting (“XGBoost” in English).

The tests of the Applicant have allowed proving that the device 2 allows obtaining very satisfactory results, even with a quite restricted training base. In this training base, the ECG and CS signals have been associated with AT origin areas according to the following distribution: Left auricle (47), Right auricle (7), Septum (158), Lateral (24). Thus, the results obtained with this training base are summarised in the table hereinbelow:

| | | | Number in |
|---|---|---|---|
| Area group | Accuracy | Reminder | the test set |
| Left auricle | 92% | 80% | 15 |
| Right auricle | 0 | 0 | 3 |
| Septum | 68% | 94% | 18 |
| Lateral | 0 | 0 | 3 |

The Cohen Kappa score has been measured at 0.55, which is very favourable, and the overall prediction rate is 74%. Recall that Cohen's Kappa score measures matching between the annotations of the test set and the prediction made thereon, while taking account of the unbalance between the representation of the classes within the latter.

Thus, the device 2 allows carrying out a “high-level” determination of the origin area of an AT in real-time, which allows considerably accelerating ablation procedures in the context of AT treatment and reduces the risks of error related to the exploration of “useless” areas of the heart, i.e. areas that are not likely to be at the origin of the TA.

1. A device for processing cardiac signals, comprising:
a memory arranged to receive input data sets each comprising a plurality of P wave segments each associated with an electrocardiogram track and with an acquisition time window, and a plurality of coronary sinus signals associated with the same acquisition time window and having one or more activation sequence(s),
an extractor arranged, for a given input data set, to determine for at least some P wave segments of the given input data set:
a wave polarity profile type,
at least one extremum feature of a type selected from a group of types comprising the number of positive local extrema, the number of negative local extrema, the positive prominence maximum and the negative prominence maximum, and
at least one integral value of these P wave segments,
and to associate the resultant data into a group of track P wave data as a function of the electrocardiogram track with which each P wave segment is associated based on which said resultant data have been calculated,
the extractor being further arranged to determine, for each group of track P wave data, a set of track P wave features comprising a polarity profile type, a data set extremum feature value for each calculated extremum feature type and an integral value determined based on the data of the corresponding group of track P wave data,
the extractor being further arranged to determine activation times in at least some of the activation sequences of the coronary sinus signals of the given input data set and to deduce a set of time values therefrom, and to return a set of data set features comprising on the one hand the set of time values and, on the other hand, the sets of track P wave features, and
a machine-learning based locator using decision trees arranged to receive a set of data set features as input, and to return a cardiac region identifier as output.

2. The device according to claim 1, wherein the locator is arranged to implement a random-forest classifier.

3. The device according to claim 1, wherein the extractor is arranged to determine a data set extremum feature value indicating an absence of determination for a non-calculated extremum feature type, and to return a set of data set features comprising a data set extremum feature value for each extremum feature type.

4. The device according to claim 1, wherein the extractor is arranged to determine a data set extremum feature value for each type of the group of types.

5. The device according to claim 1, wherein the extractor is arranged, for a given set of track P wave features, to determine the polarity profile type while retaining the predominant wave polarity profile type in the corresponding group of track P wave data.

6. The device according to claim 1, wherein the extractor is arranged, for a given set of track P wave features, to determine the data set extremum feature value for each calculated extremum feature type and the integral value based on the average of these values in the corresponding group of track P wave data.

7. The device according to claim 1, wherein the extractor is arranged to calculate the set of time values based on the difference between the activation times of the activation sequences of the coronary sinus signals.

8. The device according to claim 1, wherein the locator is arranged to receive as input a set of data set features comprising 9 sets of track P wave features, and a set of time values comprising 4 values.

9. A method for processing cardiac signals comprising the following operations:
a) receiving input data sets each comprising a plurality of P wave segments each associated with an electrocardiogram track and with an acquisition time window, and a plurality of coronary sinus signals associated with the same acquisition time window and having one or more activation sequence(s),
b) for a given input data set, for at least some of the P wave segments of the given input data set:
b1) determining a wave polarity profile type,
b2) determining at least one extremum feature of a type selected from a group of types comprising the number of positive local extrema, the number of negative local extrema, the positive prominence maximum and the negative prominence maximum, and
b3) determining at least one integral value of these wave segments P,
b4) associating the data resulting from the operations b1) to b3) into a group of track P wave data as a function of the electrocardiogram track with which each P wave segment is associated based on which said resultant data have been calculated,
b5) determining, for each group of track P wave data, a set of track P wave features comprising a polarity profile type, a data set extremum feature value for each calculated extremum feature type and an integral value determined based on the data of the corresponding group of track P wave data,
b6) determining activation times in at least some of the activation sequences of the coronary sinus signals of the given input data set and to deduce a set of time values therefrom,
b7) returning a set of data set features comprising on the one hand the set of time values of the operation b6), and, on the other hand, the sets of track P wave features of the operation b5), and
c) providing a set of data set features obtained in step b7) as input of a machine-learning based locator using decision trees, returning a cardiac region identifier as output.

10. The method according to claim 9, wherein the operation b2) comprises determining a data set extremum feature value for each type of the group of types.

11. The method according to claim 9, wherein the operation b5) comprises, for a given set of track P wave features, determining the polarity profile type while retaining the predominant wave polarity profile type in the corresponding group of track P wave data.

12. The method according to claim 9, wherein the operation b5) comprises, for a given set of track P wave features, determining the data set extremum feature value for each calculated extremum feature type and the integral value based on the average of these values in the corresponding group of track P wave data.

13. The method according to claim 9, wherein the operation b6) comprises calculating the set of time values based on the difference between the activation times of the activation sequences of the coronary sinus signals.

14. A computer program comprising instructions to execute the method according to claim 9 when said computer program is implemented by a computer.

15. A data storage medium on which the computer program according to claim 14 is recorded.
